(19) United States
(12) Patent Application Publication
(10) Pub. No.: US 2008/0044501 A1
(43) Pub. Date: Feb. 21, 2008

(54) Fermented soybean products and methods of producing them

(75) Inventors: Chan Ho Lee, Bucheon (KR); Won Tak Cho, Seoul (KR); Sung Woo Kim, Lubbock, TX (US)

Correspondence Address: JONES DAY, 555 SOUTH FLOWER STREET FIFTIETH FLOOR, LOS ANGELES, CA, 90071, US

(73) Assignee: Genebiotech Co. Ltd.

(21) Appl. No.: 11/506,057

(22) Filed: Aug. 16, 2006

(51) Int. Cl. A61K 36/48; A23L 1/36; C12P 21/06; C12N 1/20

(52) U.S. Cl. 424/757; 435/68.1; 426/44; 435/252.31

(57) ABSTRACT

Disclosed are fermented soybean protein products with enhanced enzyme productivity and methods for producing them by solid fermentation using Bacillus subtilis GR-101 and additionally Aspergillus oryzae GB-107 (KCTC 10258BP). A novel Bacillus subtilis GR-101 strain (KCCM 10673P), which has excellent protease productivity in soybean meal, is also disclosed. Fermentation using Bacillus subtilis GR-101 strain (KCCM 10673P) producing protease combined with Aspergillus oryzae GB-107 (KCTC 10258BP) having amylase activity can remove trypsin inhibitors known as anti-nutritional factors and thus can provide soybean peptides with high digestion ratio and excellent enzyme activities. Feed comprising the soybean peptides as active component and capable of improving productivity of livestock and food for human comprising the soybean peptides supply is further disclosed.

### FIELD OF THE INVENTION

The present invention relates to novel fermented soybean protein products and methods of making fermented soybean protein with enhanced enzyme productivity for superior digestibility and nutritional value.

### BACKGROUND OF THE INVENTION

Soybeans have traditionally been an important source of plant-derived protein for human consumption. Soy proteins have been reported to benefit human health by lowering cholesterol, reducing the risks of breast cancer, and atherosclerosis. Soybean meal is also the most commonly used protein source in the animal feed industry. High protein content and wide availability make soybean meal a good source of protein in animal diets. However, consuming improperly processed or uncooked soybeans can be harmful because of anti-nutritional compounds in soybeans, such as trypsin inhibitors, lectins, flatulence-producing compounds, etc. Use of soybean meal is mostly limited to adult animals because of inefficient digestibility of soy proteins by young animals. Use of soybean in infant foods may be even more problematic because of the high susceptibility of infants to anti-nutritional compounds. Attempts have thus been made to process soybean protein to increase its digestibility and nutritional value.

Current methods for chemical processing of soybean protein are cost-intensive and often cause degeneration of soybean proteins, decreased solubility and loss of essential amino acids such as lysine and methionine. Additionally, due to the high production cost of known methods for processing soybean protein, it is currently not feasible to supply significant amounts of soybean protein to livestock feed, which requires low production cost. Further, known chemical processing methods include heat treatment, chemical treatment and heat drying steps and thus typically cause degeneration of protein and a loss of water-soluble amino acids, resulting in a protein content with low solubility. To prevent severe degeneration of protein, heat treatment in such conventional methods is sometimes carried out at lower temperatures. However, this results in the incorporation of a significant amount of trypsin inhibitors, which are anti-nutritional factors and reduce the digestibility of the soybean protein. It would thus be highly desirable to develop materials and techniques for processing soybean protein products that reduce the harmful and anti-nutritional properties of soy meal, making soybean protein available as an abundant, affordable, and high-quality source of nutrition for humans, including infants, and livestock, including young animals.

### SUMMARY OF THE INVENTION

The present invention provides highly nutritional and easily digested soybean protein products comprising protease- and amylase producing microorganisms. In one embodiment the fermented soybean protein product comprises Bacillus subtilis and Aspergillus oryzae microorganisms. In preferred embodiments, the fermented soybean protein product has an initial water content of about 40-50%, as it has been found that this water content can optimize the protease and amylase activity of the microorganisms.

In another embodiment of the present invention, the protease producing microorganism is a Bacillus subtilis strain selected for its ability to produce protease. In preferred embodiments, the protease-producing Bacillus subtilis strain is GR-101 (KCCM 10673P). In yet another embodiment of the present invention, the amylase-producing microorganism is an Aspergillus oryzae strain, selected for its ability to produce amylase. In preferred embodiments, the amylase-producing Aspergillus oryzae strain is GB-107 (KCTC 10258BP).

In preferred embodiments, the B. subtilis strain produces at least 35 U/g protease, and the A. oryzae at least 100 U/g amylase. In more preferred embodiments, the B. subtilis strain produces about 70 U/g protease, and the A. oryzae about 300 U/g amylase.

Thus, the present invention provides microorganisms isolated from traditional soybean malt and selected for protease and amylase production. It should be noted that the specific strains of microorganisms disclosed herein are mere preferred embodiments and that the present disclosure enables the skilled artisan to isolate and select many equivalent microorganisms, all of which are within the scope of the instant invention.

In preferred embodiments, the present invention provides methods of producing fermented soybean protein with enhanced enzyme productivity, the method comprising adding to soybean meal a Bacillus subtilis strain having protease activity and Aspergillus oryzae strain having amylase activity. In other preferred embodiments, the Bacillus subtilis and Aspergillus oryzae strains are added to the soybean meal in a ratio of 1:1.

Also provided are food products comprising solid fermented soybean protein obtained by the methods disclosed herein. In a further preferred embodiment, the present invention provides methods of increasing the digestibility and protein content of soybean paste. The methods provided herein comprise the steps of isolating and selecting a protease-producing microorganism from soybean malt, isolating and selecting an amylase-producing microorganism from soybean malt, adding both microorganisms to soybean meal and fermenting the soybean meal with the protease- and amylase-producing microorganisms.

In preferred embodiments, the present invention provides inoculation kits for the fermentation of soybean protein product comprising protease- and amylase-producing microorganisms. In more preferred embodiments, the protease-producing microorganism is a Bacillus subtilis strain and the amylase-producing microorganism is an Aspergillus oryzae strain. In preferred embodiments, the inoculation kit contains a Bacillus subtilis strain and Aspergillus oryzae strain in a ratio of 1:1.

Thus, the present invention relates to methods of producing fermented soybean protein with enhanced enzyme productivity for superior digestibility and nutritional value. In one embodiment, the methods comprise production of soybean protein by solid-state fermentation using Bacillus subtilis GR-101 and Aspergillus oryzae GB-107 and the products obtained thereby. More particularly, the present invention relates to methods of preparing fermented soybean peptides, which methods comprise isolating and selecting a Bacillus subtilis strain with excellent protease productivity and inoculating the strain into a culture medium in which soybean meal is dipped at low temperature, followed by fermentation, to enhance enzyme productivity. The present invention further comprises culturing an Aspergillus oryzae strain with excellent amylase productivity in combination with the Bacillus subtilis strain to concentrate soybean protein and to remove trypsin inhibitors and oligosaccharides such as raffinose and stachyose. The present invention also relates to livestock feed and food for human consumption incorporating the soybean peptides fermented by the same method.

In one embodiment, the present invention provides a method for processing fermented soybean protein with enhanced enzyme productivity by solid fermentation using Bacillus subtilis GR-101 and Aspergillus oryzae GB-107 (KCTC 10258BP). The present invention also provides a novel Bacillus subtilis GR-101 strain (KCCM 10673P), which has excellent protease productivity in soybean meal. According to the present invention, fermentation using Bacillus subtilis GR-101 strain (KCCM 10673P) having excellent protease productivity combined with Aspergillus oryzae GB-107 (KCTC 10258BP) having excellent amylase activity can remove trypsin inhibitors known as anti-nutritional factors and thus can provide soybean peptides with high digestion ratio and excellent enzyme activities suitable for human consumption. Additionally, the soybean peptides obtained according to the present invention can be used as active component in livestock feed, thereby improving productivity of livestock.

### BRIEF DESCRIPTION OF THE DRAWINGS

FIG. 1 shows the result of FPLC analysis for subfractionation of fermented soybean protein according to the present invention

### DETAILED DESCRIPTION OF THE PREFERRED EMBODIMENTS

In order to process soybean protein more efficiently, the present invention provides for biological processing of fermented soybean peptides. While it is known that by isolating and selecting a microorganism, such as Aspergillus oryzae, for its ability to produce amylase, soybean meal can be cultured with a microorganism to remove carbohydrates and anti-nutritional factors including trypsin inhibitors and oligosaccharides such as raffinose and stachyose to produce fermented soybean peptides, the resulting protein is still not sufficiently hydrolyzed to be easily digested.

Thus, in one aspect the present invention provides a method of processing fermented soybean peptides comprising culturing a microorganism with excellent amylase activity in combination with a novel strain that exhibits excellent protease activity. The method improves the digestibility of fermented soybean peptides.

Accordingly, it is an object of the present invention to provide a novel microorganism strain with excellent protease productivity in order to hydrolyze the protein into smaller, more easily digested peptides.

The present invention thus provides highly nutritional and easily digested soybean protein products comprising protease- and amylase producing microorganisms. In one embodiment the fermented soybean protein product comprises Bacillus subtilis and Aspergillus oryzae microorganisms. In preferred embodiments, the fermented soybean protein product has an initial water content of about 40-50%, as it has been found that at this water content the protease and amylase activity of the microorganisms are optimized.

In another embodiment of the present invention, the protease producing microorganism is a Bacillus subtilis strain selected for its ability to produce protease. In preferred embodiments, the protease-producing Bacillus subtilis strain is GR-101 (KCCM 10673P). In yet another embodiment of the present invention, the amylase-producing microorganism is an Aspergillus oryzae strain, selected for its ability to produce amylase. In preferred embodiments, the amylase-producing Aspergillus oryzae strain is GB-107 (KCTC 10258BP).

Thus, the present invention provides microorganisms, preferably isolated from traditional soybean malt, selected for protease and amylase production. It should be noted that the specific strains of microorganisms disclosed herein are mere preferred embodiments and that persons of skill in the art, by following the present disclosure, can isolate and select many equivalent microorganisms suitable for the purposes of the instant invention. The advantage of isolating the microorganisms from traditional Korean soybean malt is that such microorganisms are already adapted to low moisture and high salt environments where other microorganisms are unable to grow. This eliminates an additional selection step and makes the isolated microorganisms well suited for use in the solid fermentation of soybean meal of the present invention.

In preferred embodiments, the present invention provides methods of producing fermented soybean protein with enhanced enzyme productivity, the method comprising adding to soybean meal a Bacillus subtilis strain producing protease and Aspergillus oryzae strain having amylase activity. In other preferred embodiments, the Bacillus subtilis and Aspergillus oryzae strains are added to said soybean meal in a ratio of 1:1.

Also provided are food products comprising solid fermented soybean protein obtained by the methods disclosed herein. In a further preferred embodiment, the present invention provides methods of increasing the digestibility and protein content of soybean paste. The methods provided herein comprise the steps of isolating and selecting a protease-producing microorganism from soybean malt, isolating and selecting an amylase-producing microorganism from soybean malt, adding both microorganisms to soybean meal and fermenting the soybean meal with the protease- and amylase-producing microorganisms.

In preferred embodiments, the present invention provides inoculation kits for the fermentation of soybean protein product comprising protease- and amylase-producing microorganisms. In more preferred embodiments, the protease-producing microorganism is a Bacillus subtilis strain and the amylase-producing microorganism is an Aspergillus oryzae strain. In preferred embodiments, the inoculation kit contains a Bacillus subtilis strain and Aspergillus oryzae strain in a ratio of 1:1.

It is yet another object of the present invention to provide livestock feed and food prepared from the fermented soybean protein, in which protein decomposition and anti-nutritional factors are minimized, obtained by using the above methods.

According to an aspect of the present invention, there is provided a method comprising the steps of: selecting a Bacillus subtilis strain with excellent protease activity; determining adequate fermentation conditions of Bacillus subtilis and Aspergillus oryzae so as to conform to a solid fermentation process; determining variations in enzyme productivity, protein level and anti-nutritional factors with the lapse of time during solid fermentation; and determining amino acid digestion ratio and fractions of fermented soybean protein by using the fermented soybean protein obtained from the preceding steps. In a preferred embodiment, the Bacillus subtilis strain is isolated from Korea traditional soybean malt, which ensures that it is adapted to low moisture and high salt environments. In preferred embodiments of the invention, the Bacillus subtilis strain is GR-101 and the Aspergillus oryzae strain is GB-107.

According to another aspect of the present invention, the Bacillus subtilis has a protease activity of 35 U/g or greater, and the Aspergillus oryzae has an amylase activity of 100 U/g or greater. The measured enzyme activities of the microorganisms may vary according to the assay and fermentation process used. Another aspect of the invention is the ability of the microorganism to remove ammonia, to produce low molecular weight peptides from the raw materials, and to maintain its own activity when grown together with another microorganism.

According to yet another aspect of the present invention, there is provided a novel Bacillus subtilis GR-101 strain (KCCM 10673P) with excellent protease productivity. In preferred embodiments, the B. subtilis strain is isolated from traditional soybean malt.

According to still another aspect of the present invention, there is provided a method for processing fermented soybean protein with enhanced protease productivity, characterized in that soybean meal having an initial water content of about 40-50% is used as culture medium, and Bacillus subtilis GR-101 strain (KCCM 10673P) with excellent protease productivity is mixed with Aspergillus oryzae GB-107 (KCTC 10258BP) in a ratio of 1:1.

According to yet another aspect of the present invention, there is provided livestock feed comprising the solid-state fermented soybean protein obtained by the above method as active component.

Reference will now be made in detail to the preferred embodiments of the present invention. It is to be understood that the following examples are illustrative only and the scope of the present invention is not limited thereto.

### EXAMPLES

### Example 1

### Isolation and Identification of Strain with Excellent Protease Productivity

Bacillus subtilis with excellent protease productivity useful for microorganisms in livestock feed was isolated from traditional soybean malt by converting high-molecular weight soybean protein into low-molecular weight peptides via fermentation with microorganisms. 1 g of soybean malt was suspended in 10 mL of sterilized saline so that it was diluted in a ratio of 1×10⁻¹ to 1×10⁻⁵. 100 μg/mL of the dilution obtained thereby was applied to a MYP culture medium (Beef extract 1 g/L, peptone 10 g/L, D-mannitol 10 g/L, sodium chloride 10 g/L, Phenol Red 0.025 g/L), followed by incubation at 37° C. for 24 hours. After the incubation, colonies appearing on the solid medium were examined with a microscope for isolation, and such isolated strains were cultured in a SK agar medium (skim milk 10 g/L, NaCl 0.24 g/L, yeast extract 0.75 g/L, anhydrous potassium phosphate (KH₂PO₂) 1.24 g/L, magnesium sulfonate (MgSO₄.H₂O) 80 mg/L, zinc sulfate (ZnSO₄.7H₂O) 0.7 mg/L and agar 15 g/L, pH 7.5) at 37° C. for 16 hours. Then, strains with a large decomposed ring of skim milk were preliminarily selected. The strains with a large decomposed ring were immersed in water to a water content of 40%, inoculated into sterilized soybean meal as culture medium, followed by incubation at 37° C. for 16 hours, and then a strain having a high protease titer and causing the largest variation in crude protein content of soybean meal was finally selected. Such final strain was examined with an optical microscope and subjected to gram staining. Identification of the strain was performed by the Korean Culture Center of Microorganisms (KCCM) through the 16S rDNA sequence. As a result, the finally selected strain was identified as Bacillus subtilis. The strain was denominated as Bacillus subtilis GR-101 and was deposited in the Korea Research Institute Bioscience & Biotechnology as the deposition No. KCCM 10673P. Table 2 shows the identification of the unknown microorganism as a new strain of B. subtilis based on a profile of the microorganism's ability to utilize different types of carbohydrate substrates.

TABLE 2
Biochemical Characteristics and Metabolic Characteristics for
Carbohydrate Availability of Bacillus subtilis GR-101

| No. | Compound | Result |
|---|---|---|
| 0 | Control | − |
| 1 | Glycerol | + |
| 2 | Erythritol | − |
| 3 | D-arabinose | − |
| 4 | L-arabinose | + |
| 5 | Ribose | + |
| 6 | D-xylose | + |
| 7 | L-xylose | − |
| 8 | Adonitol | − |
| 9 | β-methyl-D- | − |
| | xylose | |
| 10 | Galactose | − |
| 11 | Glucose | + |
| 12 | Fructose | + |
| 13 | Mannose | + |
| 14 | Sorbose | − |
| 15 | Rhamnose | − |
| 16 | Dulcitol | − |
| 17 | Inositol | − |
| 18 | Mannitol | + |
| 19 | Sorbitol | + |
| 20 | α-methyl-D- | − |
| | mannoside | |
| 21 | α-methyl-D- | + |
| | glucoside | |
| 22 | N-acethyl- | − |
| | glucosamine | |
| 23 | Amygdalin | + |
| 24 | Arbutin | + |
| 25 | Esculin | + |
| 26 | Salicin | + |
| 27 | Celobiose | + |
| 28 | Maltose | + |
| 29 | Lactose | − |
| 30 | Melibiose | − |
| 31 | Sucrose | + |
| 32 | Trehalose | + |
| 33 | Inulin | + |
| 34 | Melezitose | − |
| 35 | Raffinose | − |
| 36 | Starch | + |
| 37 | Glycogen | + |
| 38 | Xylitol | − |
| 39 | Gentiobiose | + |
| 40 | D-turanose | − |
| 41 | D-lyxose | − |
| 42 | D-tagatose | − |
| 43 | D-fucose | − |
| 44 | L-fucose | − |
| 45 | D-arabitol | − |
| 46 | L-arabitol | − |
| 47 | Gluconate | − |
| 48 | 2-keto- | − |
| | gluconate | |
| 49 | 5-keto- | − |
| | gluconate | |

### Example 2

### Determination of Optimized Fermentation Conditions of Bacillus Subtilis GR-101 and Aspergillus Oryzae GB-107 (KCTC 10258BP) Suitable for Solid Fermentation of Soybean Meal

Bacillus subtilis GR-101 and Aspergillus oryzae GB-107 (KCTC 10258BP) were examined for their optimized fermentation conditions depending on water content upon fermentation of soybean meal. To perform this, crude protein content, cell count of microorganisms and enzyme activity were examined.

First, soybean meal having an initial water content of 13% was adjusted to a water content of 30%, 40%, 50% or 60%. Next, 500 g of each soybean meal sample was introduced in a stainless steel sieve and the sieve was covered with aluminum foil, followed by cooking at 75° C. for 30 minutes and cooling to 30° C. Then, seeds of Bacillus subtilis GR-101 and Aspergillus oryzae GB-107 (KCTC 10258BP) were taken each in an amount of 1% per 500 g soybean meal, each containing 1×10⁹ and 5×10⁸/ml microorganisms) and then sprayed to the soybean meal having a water content of 30%, 40%, 50% or 60%, followed by mixing uniformly. The seeds were cultured in an incubator at 33° C. for 36 hours and dried in a dryer at 50° C. to a water content of 8%. Then, each sample was examined for amylase activity, protease activity and crude protein content. Herein, in order to determine the enzyme activity, 10 g of the soybean meal medium sample having a water content of 30%, 40%, 50% or 60% was milled in a mortar and introduced in a mass flask. After this, the mass flask was filled with distilled water to a height of 100 mL, agitated for 30 minutes and left for 2 hours. The sample was subjected to centrifugal separation (10,000×g, 4° C.) and the supernatant was used for measuring the enzyme activity.

More particularly, amylase activity was determined from the whole reducing sugar, which is prepared by using soluble starch (1% in 10 mM sodium phosphate buffer, pH 7.0) as substrate, by the DNS (dinitrosalicylic acid) method (see, [Miller, Use of dinitrosalicylic acid reagent for determination of reducing sugar. Anal. Chem. 31: 426-428, 1959]. 1 U (unit) of enzyme is defined as the amount of enzyme capable of isolating 1 μmol of the product per minute.

Protease activity was determined from tyrosine formed after the enzymatic reaction using casein (20 mM, 0.7% in Tris-HCl buffer, pH 8.0) as substrate, by the Folin method (see, [Park, Thesis Collection of Graduate School Chun-Buk University 4: 101, 1978]). 1 U (unit) of enzyme is defined as the amount of enzyme capable of isolating 1 μmole of the product per minute.

The optimized condition for growth of microorganisms depending on water content of the culture medium used in culture of the combination of strains are shown in the following Table 3.

TABLE 3
Variations in Amylase Productivity, Protease Productivity and Crude Protein by
Fermentation using Bacillus subtilis GR-101 Combined with Aspergillus oryzae
GB-107 (KCTC 10258BP) Depending On Water Content of Soybean Meal

| | | | | | Crude Protein |
|---|---|---|---|---|---|
| Initial Water | | Aspergillus | | | Content (%) |
| Content of | Bacillus | oryzae Cell | | | (with correction |
| Soybean Meal | subtilis Cell | Count | Amylase | Protease | of Water Content |
| (%) | Count (CFU/g) | (CFU/g) | (U/g) | (U/g) | of 10%) |
| 30 | 8.0 ± 0.8 × 10³ | 1.0 ± 0.4 × 10⁵ | 45.3 ± 0.7 | 5.3 ± 0.5 | 51.9 ± 1.0 |
| 40 | 5.5 ± 0.3 × 10⁷ | 5.1 ± 0.3 × 10⁷ | 235.5 ± 1.2 | 57.9 ± 0.8 | 56.9 ± 0.5 |
| 50 | 8.8 ± 0.3 × 10⁷ | 9.2 ± 0.3 × 10⁶ | 185.1 ± 0.9 | 59.2 ± 0.6 | 55.7 ± 0.4 |
| 60 | 4.4 ± 0.3 × 10⁸ | 2.1 ± 0.3 × 10⁶ | 51.3 ± 0.4 | 63.5 ± 1.2 | 53.8 ± 0.9 |

Cell count of Bacillus subtilis GR-101 increased in proportion to the water content, and protease productivity increased accordingly. However, Aspergillus oryzae GB-107 (KCTC 10258BP) showed the highest cell count and amylase activity at a water content of 40% during its fermentation combined with Bacillus subtilis GR-101. Additionally, crude protein content showed the highest level at a water content of 40%. Therefore, the optimum of water content for fermentation using Bacillus subtilis GR-101 combined with Aspergillus oryzae GB-107 (KCTC 10258BP) was 35%-45%.

Although the method according to the present invention results in an increase of crude protein content by about 0.5 to 1% compared to known methods, it provides protease activity increased by 9 times, and thus further enhances hydrolysis of soybean protein and facilitates digestion and absorption of the protein.

TABLE 4

| | Cell Count (CFU/g) | | Enzyme Titer (U/g) | | |
|---|---|---|---|---|---|
| | A. oryzae | B. subtilis | Amylase | Protease | CP(%) |
| Known Methods | 9.3 ± 1.0 × 10⁷ | — | 348.3 ± 1.5 | 6.4 ± 0.5 | 56.0 ± 0.5 |
| Present Invention | 5.5 ± 0.3 × 10⁷ | 5.1 ± 0.3 × 10⁷ | 235.5 ± 1.2 | 57.9 ± 0.8 | 56.9 ± 0.5 |

The following Table 5 shows the optimized inoculation ratio of each strain of Bacillus subtilis GR-101 and Aspergillus oryzae GB-107 (KCTC 10258BP).

TABLE 5
Optimized Ratio of Bacillus subtilis GR-101 and Aspergillus oryzae GB-107
(KCTC 10258BP) for Combined Culture Thereof

| | Addition Level | | | | |
|---|---|---|---|---|---|
| | | | | Aspergillus | |
| | | | Aspergillus | oryzae | Aspergillus |
| | | Bacillus | oryzae 80% + | 50% + Bacillus | oryzae 20% + |
| | Aspergillus | subtilis | Bacillus | subtilis | Bacillus |
| Analysis Item | oryzae 100% | 100% | subtilis 20% | 50% | subtilis 80% |
| Water Content (%) | With correction of water content of 10% | | | | |
| Crude Protein (%) | 52.15 | 53.65 | 53.85 | 56.50 | 53.90 |
| Amylase (U/g) | 305.2 | 36.5 | 98.5 | 210.5 | 78.5 |
| Protease (U/g) | 17.5 | 77.8 | 25.2 | 62.5 | 70.5 |

When fermentation was carried out by adjusting soybean meal having a water content of 46% to a water content of 40%, the optimum of the combination ratio of Bacillus subtilis GR-101 and Aspergillus oryzae GB-107 (KCTC 10258BP) was 50:50, as demonstrated by the highest increase in crude protein content. Additionally, when Bacillus subtilis GR-101 was combined with Aspergillus oryzae GB-107 (KCTC 10258BP) in a ratio of 50:50, the best result was obtained in terms of enzyme titer.

### Example 3

### Determination of Enzyme Activity

A number of microorganisms were isolated from traditional Korean soybean paste (Mae-Joo) and tested for amylase and protease activity. The following table shows the actual value of amylase and protease activity of the selected microorganisms.

| | Amylase (U/g) | Protease (U/g) |
|---|---|---|
| A. oryzae GB-107 | 300 and greater | 15 and greater |
| B. subtilis GB-101 | 30 and greater | 70 and greater |

Protease activity was determined by the following method:

### Assay for Proteolytic Activity using Azocasein

Azocasein is a chemically modified protein that was designed as a substrate for quantitative assays of proteolytic enzyme activity. It is prepared by chemically adding sulfanilamide groups to the milk protein casein. The sulfanilamide groups, which are orange, are covalently linked to the peptide bonds. Reaction mixtures are set up using azocasein as the substrate. They are then incubated under the conditions chosen for the experiment. As the proteolytic enzyme hydrolyzes peptide bonds, shorter peptides and amino acids are liberated from the chain.

Following incubation, trichloroacetic acid (TCA) is added to stop the enzyme and precipitate (by denaturation) macromolecules, including the enzyme and undigested azocasein. These are removed from the reaction mixture by centrifugation. Very short peptide chains and free amino acids liberated by the proteolytic enzyme are not precipitated by the TCS, and thus remain in solution. The greater the extent of proteolysis, the greater the number of short peptide chains and free amino acids in solution, and thus, the more intense the orange color of the solution. The intensity of the color, measured spectrophotometrically, is used to determine the relative activity of the proteolytic enzyme. The assay has been in use since 1949 (Tomarelli, R. M., J. Charney, and M. L. Harding. 1949. The use of azoalbumin as a substratein the colorimetric determination of peptic and tryptic activity. Jour. Lab. and Clin. Med. 34:428-433.)

Assay for Amylase Activity

Amylase activity was measured by the dinitrosalicylic acid (DNS) method (Rick, W. and H. P. Stegbauer. 1974. α-Amylase measurement of reducing groups. In H. V. Bergmeyer (ed.), Methods of Enzymatic Analysis, 2nd edn., vol. 2, Academic Press, New York.). One unit of amylase activity was defined as the amount of enzyme hydrolyzing 1 μmol of glucose equivalents of starch in one minute under the assay condition. 5 ml of soluble starch solution, 200 μl enzyme solution and 4.8 ml distilled water were mixed and reacted at 37° C. for 30 min, 3 ml of DNS solution was then added. The reaction was stopped by boiling for 5 min. The absorbance was measured at 540 nm.

An alternative amylase activity is that developed by Smith and Roe, J. Biol. Chem., 179, 53 (1949), as modified by Manning and Campbell (J. Biol. Chem, 236:11, 2952-2957). Briefly, a 1% solution of Lintner soluble starch is prepared in 0.1 M acetate buffer, pH 4.6. To each of two test tubes were added 5.0 ml of the buffered starch substrate, 3.0 ml of 0.1 M acetate buffer (pH 4.6), and 1.0 ml of 0.5 M CaCl₂. A third tube served as a reagent blank. The tubes were incubated at 65° C. until equilibrated. To one tube was added 1.0 ml of enzyme and, after a 10-minute reation time at 65° C., 2.0 ml of 1 N HCl were added to each tube. Enzyme (1.0 ml) was then added to the reagent blank and the undigested starch control tube. After thorough mixing, 0.2 ml of each of the three tubes was placed in 50-ml volumetric flasks containing o.5 ml of 1 N HCl and 40 ml of distilled water. Color was developed in each flask by the addition of 0.1 ml of an iodine solution (3.0% KI and 0.3% I₂). The flasks were brought to volume and thoroughly mixed. The resultant blue solutions were decanted into colorimeter tubes and absorbancy read at 620 nm with a Bausch and Lomb Spectronic 20 colorimeter. Activity was calculated by the equation:

$$\frac{AbC - AbD}{AbC} \times mg of starch initially present = mg of starch hydrolyzed$$

in which AbC is the absorbancy of the control and AbD is the absorbancy of the digest. ONe unit of α-amylase is that amount of protein which will hydrolyze 10 mg of starch per minute under the specified conditions. Specific activity is expressed as units per mg. of protein.

### Example 4

### Variations in Enzyme Activity, Crude Protein Content and Anti-Nutritional Factors During Large-Scale Production Depending on Fermentation Conditions

After fermentation using Bacillus subtilis GR-101 combined with Aspergillus oryzae GB-107 (KCTC 10258BP) for preparation of fermented soybean peptides, protein dispersibility index (PDI) and KOH solubility index were measured in order to obtain information about amounts of anti-nutritional factors of soybean meal, including trypsin inhibitors, raffinose and stachyose and protein solubility.

For this, solid fermentation using Bacillus subtilis GR-101 combined with Aspergillus oryzae GB-107 (KCTC 10258BP) was performed in soybean meal having water content adjusted to 40%. During the fermentation, variations in enzyme activity and crude protein content were observed.

Titer of trypsin inhibitors was determined by the method according to AACC-71-10 (American Association of Cereal Chemists, 1995). Raffinose and stachyose were determined by liquid chromatography (LC). Protein dispersibility index was determined as follows: 0.5 g of a sample was introduced into 150 mL of water, and the mixture was stirred at 8,500 rpm for 10 minutes and filtered. Next, the filtrate was measured for its nitrogen content by using a kjeldahl instrument and the nitrogen content was expressed as solubility (see, Batal, et. al, Protein dispersibility index as an indicator of adequately fermented soybean meal, Polut Sci. 79: 1592-6, 2000). Then, KOH solubility was obtained as follows: 0.1 g of a sample was added to 0.2% aqueous KOH solution, and the mixture was mixed for 20 minutes and filtered. Next, the filtrate was measured for its nitrogen content by using a kjeldahl instrument and the nitrogen content was expressed as solubility (see, Parsons, et. al, Soybean protein solubility in potassium hydroxide: an in vitro test of in vivo protein quality. J Anim Sci. 69: 2918-24, 1991).

TABLE 6
Variations in Large-Scale Fermentation using Bacillus subtilis GR-101 Combined with
Aspergillus oryzae GB-107 (KCTC 10258BP) over Time

| | | | Fermentation | | | | |
|---|---|---|---|---|---|---|---|
| | Materials | Cooking | 0 hr | 12 hrs | 24 hrs | 36 hrs | After Drying |
| Crude Protein | 47.7 ± 0.2 | 47.7 ± 0.3 | 47.7 ± 0.2 | 49.2 ± 0.4 | 54.3 ± 0.6 | 56.4 ± 0.8 | 56.5 ± 0.5 |
| Content (%) | | | | | | | |
| (with correction | | | | | | | |
| of water content | | | | | | | |
| of 10%) | | | | | | | |
| Water Content | 40 ± 0.4 | 41.5 ± 0.5 | 41.0 ± 0.3 | 38.5 ± 0.5 | 35.4 ± 0.5 | 32.8 ± 0.3 | 8.4 ± 0.4 |
| (%) | | | | | | | |
| Aspergillus | — | — | 2.1 ± 0.2 × 10⁴ | 8.8 ± 0.2 × 10⁴ | 9.1 ± 0.5 × 10⁵ | 4.1 ± 0.5 × 10⁷ | 4.0 ± 0.6 × 10⁷ |
| oryzae Cell | | | | | | | |
| Count (CFU/g) | | | | | | | |
| Bacillus Subtilis | — | — | 2.3 ± 0.3 × 10⁴ | 3.1 ± 0.4 × 10⁵ | 8.5 ± 0.6 × 10⁶ | 6.1 ± 0.8 × 10⁷ | 6.5 ± 0.5 × 10⁷ |
| Cell Count | | | | | | | |
| (CFU/g) | | | | | | | |
| Amylase (U/g) | 2.0 | 2.0 | 2.1 | 59.5 | 152.4 | 210.1 | 208.3 |
| Protease (U/g) | 3.2 | 3.2 | 3.0 | 25.3 | 46.5 | 57.2 | 57.5 |
| Trypsin | 3.85 ± 0.6 | 2.10 ± 0.4 | 2.10 ± 0.3 | 1.65 ± 0.8 | 0.92 ± 0.3 | 0.67 ± 0.5 | 0.65 ± 0.4 |
| Inhibitors | | | | | | | |
| (TImg/g) | | | | | | | |

As fermentation using Bacillus subtilis GR-101 combined with Aspergillus oryzae GB-107 (KCTC 10258BP) progressed, enzyme titer and crude protein content increased. Also, enzyme production increased with the lapse of fermentation time, and particularly increased rapidly after 24 hours from the start point of the fermentation.

Additionally, trypsin inhibitor titer was reduced significantly according to fermentation degrees of Bacillus subtilis GR-101. Particularly, about 55% of trypsin inhibitor titer could be reduced by known heat treatment methods and 45% could be reduced by fermentation according to known methods. However, addition of Bacillus subtilis GR-101 enhances protease activity as a result of fermentation, so that trypsin inhibitor titer is further reduced by about 0.3 TI mg/g (0.3 mg trypsin inhibitor/g soybean meal), compared to previously known methods.

The following Table 7 shows the results of anti-nutritional factors measured after the fermentation using the novel Bacillus subtilis GR-101 according to the present invention combined with Aspergillus oryzae GB-107 (KCTC 10258BP).

TABLE 7
Anti-nutritional Factor Content after the Fermentation using
Bacillus subtilis GR-101 Combined with
Aspergillus oryzae GB-107 (KCTC 10258BP)

| | Raffinose | Stachyose | Trypsin Inhibitors |
|---|---|---|---|
| Unprocessed | 1.34 | 3.6 | 3.85 |
| Soybean | | | |
| Meal | | | |
| Present | 0 | 0 | 0.6 |
| Invention | | | |

After the fermentation using Bacillus subtilis GR-101 according to the present invention combined with Aspergillus oryzae GB-107 (KCTC 10258BP), anti-nutritional factor content was reduced significantly compared to soybean meal, and stachyose and raffinose were not detected. This indicates that fermentation using the novel Bacillus subtilis GR-101 according to the present invention combined with Aspergillus oryzae GB-107 (KCTC 102518BP) enhances protease activity, even if the above anti-nutritional factors are partially destroyed during heat treatment.

As can be seen the above results, fermentation using Bacillus subtilis GR-101 according to the present invention combined with Aspergillus oryzae GB-107 (KCTC 10258BP) under the optimized conditions can reduce the amount of anti-nutritional factors, i.e. stachyose and raffinose significantly as well as improve the protein dispersibility index and KOH solubility index, compared to previously known methods.

### Example 5

### Determination of Amino Acid Digestion Ratio and Soybean Protein Fraction in Weaned Pigs Using Bacillus Subtilis GR-101 According to the Present Invention and Aspergillus Oryzae GB-107 (KCTC 10258BP)

In order to determine amino acid digestion ratio of the fermented soybean protein according to the present invention in weaned pigs, thirty head of ternary hybridized (Yorkshire×Landrace×Duroc) weaned pigs were selected and each pig was housed in a separate metabolism cage. As control, soybean meal was used, and fermented soybean protein was measured for the amino acid digestion ratio by collecting excrete from the pigs for two days from the fourth day of the feed intake after the lapse of time needed for adaptation.

TABLE 8
Amino Acid Digestibility in Weaned Pigs Using Soybean
Meal, Bacillus subtilis GR-101 and Aspergillus oryzae
GB-107 (KCTC 10258BP)

| | | |
|---|---|---|
| | | Fermented |
| | | Soybean Protein |
| | | According |
| | | to the Present |
| Amino Acid Digestion Ratio (%) | Soybean Meal | Invention |

Essential Amino Acids

| | | |
|---|---|---|
| Arginine | 86.64 | 92.04 |
| Hystidine | 61.90 | 87.35 |
| Isoleucine | 58.49 | 86.15 |
| Leucine | 55.65 | 86.53 |
| Lysine | 60.97 | 91.03 |
| Methionine | 28.31 | 83.40 |
| Phenylalanine | 66.87 | 86.74 |
| Threonine | 66.33 | 88.53 |
| Valine | 51.76 | 88.41 |
| Total Essential Amino Acids | 59.66 | 87.80 |

Non-essential Amino Acids

| | | |
|---|---|---|
| Alanine | 42.93 | 86.00 |
| Aspartic Acid | 61.34 | 87.47 |
| Cystine | 54.03 | 86.68 |
| Glutamic Acid | 64.22 | 83.22 |
| Glycine | 47.95 | 87.90 |
| Proline | 72.93 | 89.31 |
| Serine | 64.01 | 85.12 |
| Tyrosine | 68.42 | 87.31 |
| Total Non-essential Amino Acids | 59.48 | 86.63 |

Digestibility of the total essential amino acids of the control (soybean meal) was 59.66%, while that of the soybean protein fermented by using Bacillus subtilis GR-101 and Aspergillus oryzae GB-107 (KCTC 10258BP) was 87.80%. Additionally, digestibility of the non-essential amino acids of the control (soybean meal) was 59.48%, while that of the fermented soybean protein was 86.63%, which was significantly higher compared to the control.

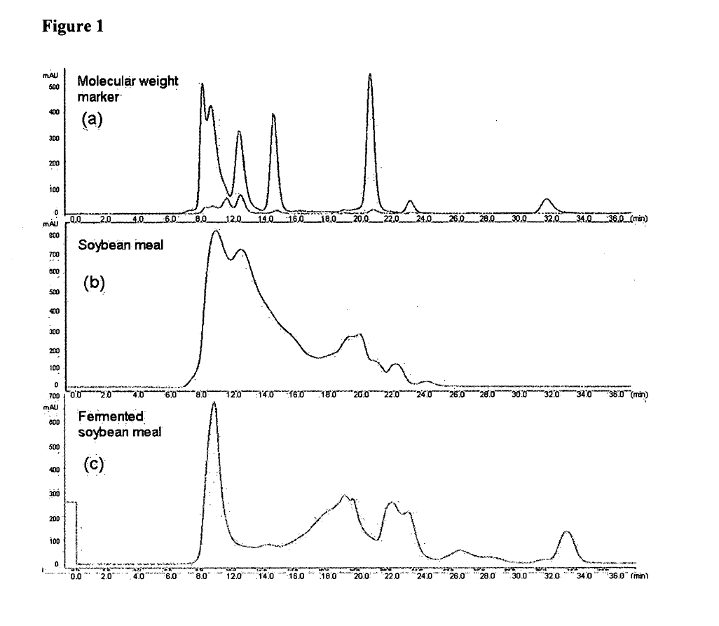

To check the peptide sub-fractionation of the fermented soybean protein according to the present invention, the acetone concentration method was used to extract protein and the extract was subjected to fast protein liquid chromatography (FPLC) equipped with a Sepadex 75 column to perform analysis for sub-fractionation of the protein. After the analysis using FPLC, constitution of the peptides are shown in FIG. 1.

FIG. 1 contains a chromatography (FPLC) of peptide distributions from Fermented Soybean Meal (FSM) and Conventional Soybean Meal (CSM). FIG. 1 contains three graphs: (a) Molecular marker, (b) CSM, and (c) FSM. The Y-axis is mAU and the X-axis is retention time (min).

FIG. 1 can be further summarized by the following table:

TABLE 1
Summary of Peptide distribution (%)of FSM and CSM by size.

| Category | MW (Da) | CSM (%) | | FSM (%) | |
|---|---|---|---|---|---|
| Large | ≧66000 | | 79.64 | | 32.00 |
| Medium | 5001–65999 | | 1.28 | | 0.00 |
| Small | ≦5000 | | 18.03 | | 67.50 |
| | | Total: | 98.95 | Total: | 99.50 |

A comparison of the FSM with the CSM reveals that the FSM processed with the present method contains significantly greater numbers of smaller sized peptides. 79.64% of the CSM is comprised of large peptides while 1.28% are medium peptides and only 18.03% are small peptides. In contrast, a look at the FSM shows that only 32% are large peptides while 0.0% are medium peptides and 67.50% are small sized peptides.

This indicates that fermentation using Bacillus subtilis GR-101 combined with Aspergillus oryzae GB-107 (KCTC 10258BP), having excellent protease titer and amylase titer, permits soybean meal to be hydrolyzed more efficiently, so that high-molecular weight protein is degraded into low-molecular weight peptides and low-molecular weight peptides predominate.

1. A fermented soybean protein product comprising a microorganism selected for protease-production and a microorganism selected for amylase production.

2. The fermented soybean product of claim 1, wherein said microorganism selected for protease production is Bacillus subtilis.

3. The fermented soybean product of claim 1, wherein said microorganism selected for amylase production is Aspergillus oryzae.

4. The fermented soybean protein product of claim 2, wherein said Bacillus subtilis produces protease in the amount of at least 35 Units per gram.

5. The fermented soybean protein product of claim 2, wherein said Bacillus subtilis produces protease in the amount of about 70 Units per gram.

6. The fermented soybean protein product of claim 2, wherein said Bacillus subtilis is GR-101 (KCCM 10673P).

7. The fermented soybean protein product of claim 3, wherein said Aspergillus oryzae produces amylase in the amount of at least 100 units per gram.

8. The fermented soybean protein product of claim 3, wherein said Aspergillus oryzae produces amylase in the amount of about 300 units per gram.

9. The fermented soybean protein product of claim 7, wherein said Aspergillus oryzae is GB-107 (KCTC 10258BP).

10. A Bacillus subtilis strain isolated from traditional Korean soybean malt which produces protease.

11. The Bacillus subtilis strain of claim 10, which produces protease in the amount of at least 35 units per gram.

12. The Bacillus subtilis strain of claim 10, which produces protease in the amount of about 70 units per gram.

13. The Bacillus subtilis strain of claim 10, wherein the Bacillus subtilis strain is GR-101 (KCCM 10673P).

14. A method of producing fermented soybean protein with enhanced enzyme productivity, the method comprising adding to soybean meal the Bacillus subtilis strain of claim 10.

15. The method of claim 14, further comprising adding to the soybean meal an Aspergillus oryzae strain having amylase activity.

16. The method of claim 15, wherein said Bacillus subtilis and said Aspergillus oryzae are added to said soybean meal in a ratio of 1:1.

17. The method of claim 16, wherein said soybean meal has an initial water content of about 40% to 50%.

18. The method of claim 14, wherein said Bacillus subtilis strain produces protease in the amount of at least 35 units per gram.

19. The method of claim 18, wherein said Bacillus subtilis strain is GR-101 (KCCM 10673P).

20. The method of claim 15, wherein said Aspergillus oryzae has an amylase activity of at least 100 units per gram.

21. The method of claim 15, wherein said Aspergillus oryzae is GB-107 (KCTC 10258BP).

22. A food product comprising solid fermented soybean protein obtained by the method of claim 14.

23. A method of increasing the digestibility and protein content of soybean paste, the method comprising:
a) selecting a protease-producing bacillus subtilis strain;
b) selecting an amylase-producing aspergillus oryzae strain;
c) adding said bacilllus and aspergillus strains to soybean meal; and
d) fermenting said soybean meal.

24. The method of claim 23, wherein said soybean meal has an initial water content of about 40-50%.

25. The method of claim 23, wherein said bacillus subtilis strain produces protease in the amount of at least 35 units per gram.

26. The method of claim 23, wherein said and aspergillus oryzae strain produces amylase in the amount of at least 100 units per gram.

27. The method of claim 23, wherein said bacillus subtilis strain is GR-101 (KCCM 10673P).

28. The method of claim 23, wherein said and aspergillus oryzae strain is GB-107 (KCTC 10258BP).

29. An inoculation kit for the fermentation of soybean protein product comprising a Bacillus subtilis strain and an Aspergillus oryzae strain.

30. The inoculation kit of claim 24, wherein said Bacillus subtilis strain and Aspergillus oryzae strain are present in a ratio of 1:1.
